(19) United States
(12) Patent Application Publication
(10) Pub. No.: US 2023/0293720 A1
(43) Pub. Date: Sep. 21, 2023

(54) Novel nanobubble that contains epilepsy medicine. The bubble polarizes in presence of the electric surge generated in the brain during an epilepsy attack and automatically discharges the medicine at the time and the site of the attack, stopping the seizure in its infancy without any external intervention. The bubble releases just enough medication into the bloodstream, significantly reducing side effects.

(71) Applicants: Sohini Banerjee, Herndon, VA (US); Sanchali Banerjee, Herndon, VA (US)

(72) Inventors: Sohini Banerjee, Herndon, VA (US); Sanchali Banerjee, Herndon, VA (US)

(21) Appl. No.: 17/581,366

(22) Filed: Jan. 21, 2022

(51) Int. Cl. A61K 47/69

(52) U.S. Cl. CPC A61K 47/6925

(57) ABSTRACT

Epilepsy is a neurological disorder, causing unprovoked electrical storms at different points in the brain, resulting in seizures, unconsciousness, and even death. The Epilepsy Foundation of America says that the goal of epilepsy medicine is “no seizures and no side effects.” However, this is difficult to achieve with current medications and procedures. The invention is a novel nanobubble containing epilepsy medicine. The bubble polarizes in presence of the electric surge generated in the brain during an epilepsy attack; it automatically discharges the medicine at the time and the site of the attack, stopping the seizure in its infancy without any external trigger. The radius and shell thickness of the nanobubble are designed for the bubble to burst above a particular threshold of electricity produced by an epileptic seizure.

### INVENTION SUMMARY

The invention is a novel nanobubble that contains epilepsy medicine. The bubble polarizes in presence of the electric surge generated in the brain during an epilepsy attack and automatically discharges the medicine at the time and the site of the attack, stopping the seizure in its infancy without any external intervention. The bubble releases just enough medication into the bloodstream, significantly reducing side effects.

### BACKGROUND

Epilepsy is a neurological disorder, causing unprovoked electrical storms at different points in the brain, resulting in seizures, unconsciousness, and even death. It impacts 65 million people worldwide of all races, genders, and ages. However, the direct cause of a seizure is seldom known, so patients take additional medication to deal with what may or may not be the seizure’s cause. Due to this, unwanted drugs become present in the body, inducing side effects. For instance, administering large amounts of medicine can result in excessive subduction of electrical activity in the brain, which slows down cognitive functions. Additionally, there is no way to predict exactly when an epileptic attack will occur, so by the time a patient takes medicine to control the seizure that has already started, it will often have already spread to other parts of the brain. Our innovation suppresses the epileptic seizure and its accompanying side effects in its infancy.

The Epilepsy Foundation of America says that the goal of epilepsy medicine is “no seizures and no side effects”. However, this is difficult to achieve. Of the 65 million epileptic patients worldwide, 39 million patients do not know the cause of their disease. For the people who are aware of the cause of their disorder, the cause can be related to genes, structural abnormalities in the brain, metabolic disruptions, irregular antibodies in the immune system, or infections. However, regardless of whether a patient knows the cause of their disease or not, there is still no way to know exactly when a seizure will occur. Consequently, the focus shifts to controlling the seizures instead of curing them.

Common epilepsy medicines reduce the probability of seizures but produce significant side effects, due to the overabundance of epilepsy medicines released into the bloodstream. Since the attack’s cause is unknown in 60% of the cases, medications cannot prevent the attack. They are also unable to prevent death in extreme circumstances, including attacks that occur during sleep. Some patients approach this challenge by relying on rescuer medicines that would quickly dissolve in the blood and act on the brain. However, by the time a patient feels the need to take the medication, it is often too late. It is not of much use during sleep either, since the patient will not be able to recognize that an attack is imminent. This may even lead to death in sleep. Regularly taking rescuer medications to prevent attacks increases the amount of medicine in the bloodstream, and hence side effects.

Our product uniquely encapsulates the medication in bubbles, releasing it only at specific r locations in the brain where the attack is developing, thus controlling seizures automatically with as little medicine as possible and reducing side effects.

### DRAWINGS

The patent or application file contains at least one drawing executed in color. Copies of this patent or patent application publication with color drawing(s) will be provided by the Office upon request and payment of the necessary fee.

### Drawing Description

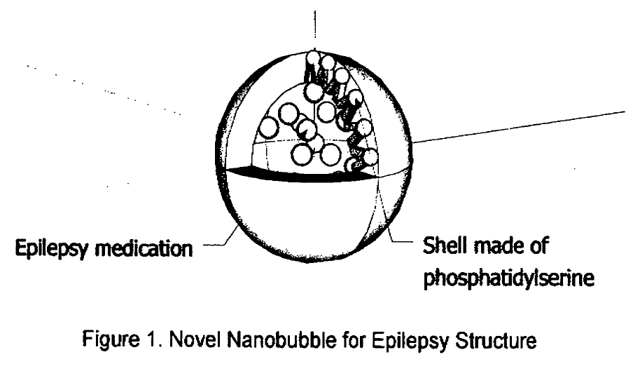

FIG. 1 The diagram shows the nanobubble to treat epilepsy.

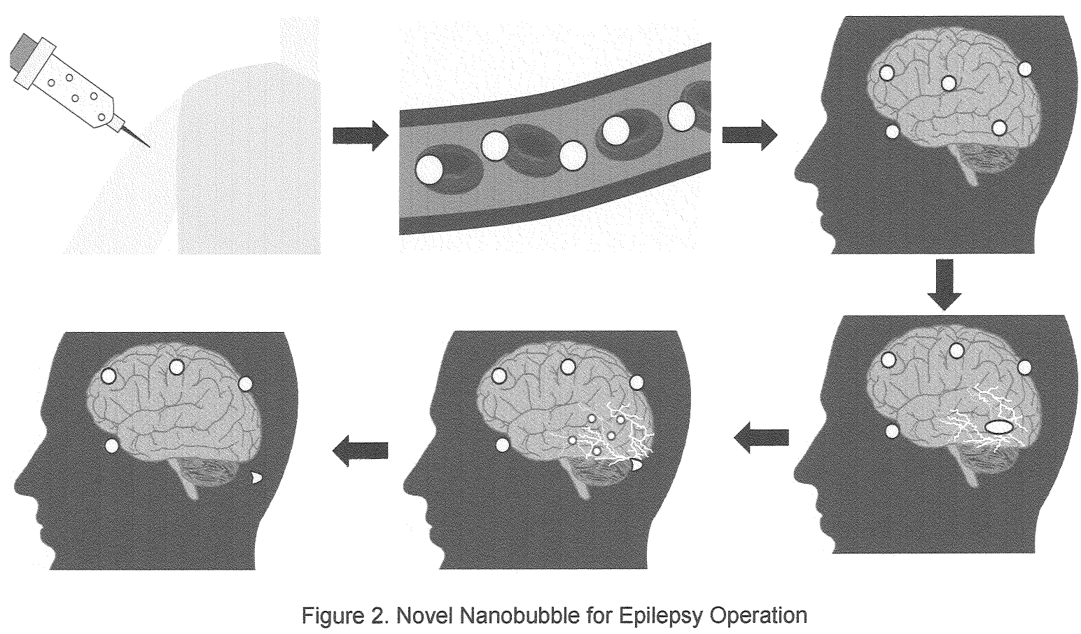

FIG. 2 The diagram shows how the nanobubble will function.

### INVENTION DETAILS

We propose a novel nanobubble that automatically reacts to a developing epilepsy attack and discharges medication to subdue the impact and save lives. A nanobubble is a bubble containing medication that is injected into a patients body, releasing its medicine once it bursts. Our nanobubbles become polarized in the presence of the electric field that develops during an epilepsy attack. The resulting electrostatic force between nerve cells and the bubble stretches the bubble, rupturing it at a certain threshold. Once it bursts, it ejects medication towards the site of the attack along the lines of the electric field between nerve cells and the bubble. The medication pacifies the nerve cells, preventing the attack from spreading to the entire brain. The Blood-Brain Barrier, the membrane separating the nerve cells and the circulating blood carrying the bubbles, is permeable enough to allow the electric field to impact the bubbles and provides passage of the medicine discharged by the bubble to reach nerve cells.

Our novel nanobubble addresses the challenges presented by epilepsy because it harnesses the unique characteristics of epilepsy to trigger the delivery of a drug at the appropriate time and location within the body, without any external intervention. By doing this, the patient’s treatment can be automatically initiated as soon as a seizure begins to develop, which ensures that it will not have already spread to other parts of the brain when medication is administered. Additionally, since it will only release its drug when there is a certain amount of electricity, our nanobubbles are guaranteed to eliminate the presence of excess medicine in the bloodstream. This removes the occurrence of serious side effects that could potentially be fatal.The bubbles that are not used to suppress a seizure are guaranteed to not burst, so injecting nanobubbles even in absence of a seizure will not cause any harm.

Epileptic patients would periodically use shots containing nanobubbles, similarly to diabetic patients, with the dosage determined by the patient’s medical history. By injecting enough quantities of nanobubbles into the bloodstream, sufficient numbers will inevitably flow to the brain. They will serve as gatekeepers at different parts of the brain, activating in response to the developing electric field of a seizure.

Nanobubbles have been used in the industry. However, currently, there is no innovation using nanobubbles to treat epilepsy. Our nanobubble is unique because it is the first nanobubble to utilize the electric properties of epilepsy to its advantage. Our nanobubble releases just enough medication at the right place and time without any external intervention by taking advantage of the electric surge in the brain generated during epilepsy attacks. There will not be unnecessary medication in the bloodstream, eliminating side effects. Other products using nanobubbles are oncology-focused and use external stimulus (ultrasound) for their products. FIG. 1 shows the structure of our nanobubble for epilepsy treatment.

The sequence of diagrams in FIG. 2 show the bubbles flowing through the bloodstream into the brain. Subsequent diagrams show the bubble stretching and rupturing in presence of the electric field produced by the epilepsy attack and releasing the medicine to prevent the attack from spreading.

### TECHNICAL PRINCIPLES UTILIZED

The conception of our novel nanobubble designed to treat epilepsy for the first time evolved from observations in our daily lives, published research in physics and life sciences, and the existence of biocompatible materials that could make up the bubble. We outline below the principles behind these observations, research, and materials.
1. Bubble Physics
2. The Effect of Electric Force on Bubbles
3. Electric Surge in the Brain
4. Material Ionization
5. Micro/Nanobubbles (MNBs)

### Bubble Physics

We observe bubbles in our daily lives. Most commonly, they have a film of soap-water enclosing air molecules. Vital to the bubble’s longevity is the concept of surface tension, the force that holds the molecules of the liquid together. It is the lower surface tension of the soap-water as compared to drinking water that allows the molecules of the soap water to stay further apart and form the bubble, instead of forming droplets. Simultaneously, the surface tension is high enough to withstand the outward pressure of the internal gas core. The forces due to surface tension and the outward pressure from the gas core are dependent on the bubble’s radius. For example, a larger bubble will be less stable compared to a smaller bubble, because of higher surface tension and lower pressure from the gas core. Both of these things make the bubble less firm. Thus, by controlling the density/composition of the gas core, the external material, and the bubble’s radius, we can control the stability and hence the longevity of our novel nanobubble within the human body.

### The Effect of Electric Force on Bubbles

An experiment with a soap bubble and a plastic bottle rubbed against hair (youtube.com/watch?v=aySWX55-xX4) shows how the bubble changes its shape and bursts when near the bottle. The electrostatic charge on the surface of the bottle forces the accumulation of charges on the side of the bubble near the bottle. The resulting electrostatic attractive force between the opposite charges of the bubble and the bottle stretches the bubble on the near side of the electric source. The strength of the electrostatic force depends on the charges of the two items (bottle and bubble) and the distance between them. If strong enough, the elongation caused by the electric force bursts the bubble, releasing the content of the bubble towards the bottle.

### Electric Surge in the Brain

The epilepsy attack may occur anywhere in the brain. It is characterized by the sudden increase in electric charges, like the accumulation of electric charges in a cloud. The surge results in subsequent propagation over different parts of the brain and the nervous system, causing the seizure. The electric field in the brain is represented by zeta-potential, the potential difference in the strength of the electric field that arises between the stationary surface and the fluid, i.e. between the neural junctions where the charges accumulate and the blood stream (sciencedirect.com/topics/pharmacology-toxicology-and-pharmaceutical-science/zeta-potential). The zeta-potential in the brain has the ability to span across the Blood-Brain Barrier (BBB), the membrane that separates the neural structures and the blood stream. Consequently, any particle flowing through the bloodstream in the brain would be influenced by the zeta-potential. Our goal is to sense if the rising zeta-potential has reached a threshold, at which point, we would neutralize it before it spreads to other areas of the brain. The threshold should be below the level at which the epileptic attack starts to spread.

### Material Ionization

The electrostatic force between the plastic bottle and the bubble is possible if opposite charges accumulate on the side of the bubble near the bottle. The bubble’s surface material should, therefore, be able to ionize in presence of the electric field produced by the bottle carrying static electricity, for the electric force to form and impact the bubble. We would select a material such that it is proven to be biocompatible and can easily ionize in presence of the electric field. For the bubble to stretch towards the site of the electric source, the bubble material should be able to ionize with an opposite charge to the electric source.

### Micro/Nanobubbles (MNBs)

Our product is built upon the principles of micro/nanobubbles (MNBs). The general composition of an MNB is a core composed of either gas or a mixture of gas and medication, surrounded by a shell of lipids, proteins, or polymers. When they are injected into the body, ultrasound imaging is often utilized to track their location. Once they reach a target point within the body, external intervention (ultrasound, heat, etc.) is used to stimulate the rapid expansion and contraction of the bubble or the elongation of the bubble. This causes the bubble to burst and release its medicine at a specific point in the body. The typical diameter of a microbubble is 1-20 micrometers (ncbi.nlm.nih.gov/pmc/articles/PMC2889676/). However, this makes it difficult for the bubbles to pass through the bloodstream and reach their target location, since they are sometimes too large. This is why our nanobubbles are more suitable for our epilepsy treatment method, since their diameter is much smaller than microbubbles, being less than 200 nanometers (moleaer.com/nanobubbles#:~:text=Nanobubbles%20have%20more%20than%20400,is%20eff ectively%20delivered%20to%20water.). The smaller size of the nanobubbles provides greater stability, allowing the nanobubbles to remain in systemic blood circulation for much longer. Our innovation is similar to typical MNBs, as it is designed to release its drug at a target location with certain stimulation. However, instead of requiring external intervention for activation, it works using the electricity generated at the site of a seizure.

Our nanobubble is the first product to utilize the characteristics of epilepsy to its advantage. While being built upon existing research, it is an innovative solution to treat epilepsy.

### BUBBLE DESIGN

FIG. 1 shows the structure of our nanobubble.

### Exterior Material Considerations

When the nanobubbles are near the brain, the electric charges and bubbles need to attract. This is because the nanobubbles will only release their medicine by bursting, so by being close to the charged region, the bubbles can easily elongate, burst, and release their drug. This also allows the seizure to be pacified at its point of origin because the bubbles will be attracted to wherever the electric surge is starting. For the bubbles and the electric charges to attract, they must be of opposite charges. According to NCBI (ncbi.nlm.nih.gov/pmc/articles/PMC4612498/), the net electric charge from the brain is positive. Therefore, the exterior of the bubbles must be negatively charged for them to attract.

### Exterior Material

Proteins, which are macromolecules composed of one or more amino acid residue chains, are highly sensitive to extreme temperatures, causing the nanobubbles’ shell to be easily destroyed. This means the drug can be ejected easily. This decreases therapeutic efficacy because the drug may be released at the incorrect location or time, inducing unwanted side effects without actually dealing with the seizure.

Lipids, which are macromolecules made of fatty acid monomers. This makes them a more reliable material to use than proteins. Lipids are also not as sensitive as proteins, but still not as stiff as synthetic biopolymers, which would make lipid-shelled nanobubbles capable of bursting at the optimal time, not too soon or too late. Additionally, they are often preferred because of their high biocompatibility.

Nanobubbles made of synthetic biopolymers, which are substances with a molecular structure of several repeated units, would be generally more rigid than lipid-coated bubbles. This makes it more difficult for the bubbles to expand and contract, so they are more resistant to bursting at the correct time. Therefore, lipids are the optimal material to use for the exterior of our novel nanobubble.

### Exterior Lipid

Phospholipids contain a phosphate head group, and have both hydrophilic and lipophilic regions. The most common phospholipid head group is the phosphate, PO 3- 4, and it is negatively charged. The phospholipid with this head group is called phosphatidylserine. This is the best lipid to use for the exterior of our nanobubble because its phosphate head group is negatively charged, making the surface of our bubble negatively charged, allowing it to be attracted to the brain’s positive charges. Additionally, because phosphatidylserine contains hydrophilic and lipophilic regions, it is capable of passing through the Blood-Brain Barrier and reaching the seizure’s electric source in the brain. Phosphatidylserine is, therefore, the best material to use for the exterior of our novel nanobubble.

### Interior Substance

The interior of our nanobubble will comprise a mixture of epilepsy medication and a certain gas. By cloaking the medicine with the shell of the bubble, the immune system will not react to the incoming drug. When medicine is injected into the bloodstream on its own and not within a bubble, the immune system often interprets it as a threat to the body and attacks it. Consequently, the target location in the patient’s body cannot receive its medicine, since the immune system attempts to remove the drug from the system. However, by cloaking the drug with a biocompatible material (phosphatidylserine), the medication will be protected from the immune system. This allows the medicine to be delivered to the target location without being diverted.

The medication must be part of a mixture with another gas, to ensure the stability of the nanobubble. For the bubble to not collapse from external pressure when passing through the bloodstream, it must contain a gas that will withstand the pressure and will not react with the medicine. The medicine itself cannot withstand the pressure unless high amounts of it are in the nanobubble, which will only make the bubble extremely heavy because the medicine is made of solid particles; the interior must have a fluid (gas) for the bubble to be stable and move easily in the bloodstream.

Using pure oxygen as the interior gas causes nanobubbles to dissolve quickly and release their drug too easily, even without much stimulation. By using a hydrophobic gas, which tends to fail to mix with water and has a high molecular weight, the stability of our nanobubbles can be improved. Gases like perfluorocarbons and sulfur hexafluoride in nanobubbles, in conjunction with oxygen, improve their longevity. However, using sulfur hexafluoride has been banned by the European Union because it has one of the highest global warming potentials of several greenhouse gases Although certain perfluorinated compounds have been banned in America, perfluorocarbon is not one of them, so it is viable According to NCBI, a mixture with 95% oxygen and 5% perfluorocarbon is the optimal proportion for the stable release of the medication within our nanobubbles.

Therefore, the interior of our nanobubbles will be a mixture of oxygen, perfluorocarbon, and epilepsy medication.

### Dimensions

The nanobubble needs to deform and rupture in presence of the electric field that would cause such a deformation. The deformation depends on the surface tension of the bubble, which depends on the bubble radius and the surface tension coefficient of the outer material of the bubble. The coefficient of surface tension of a material is a force that acts perpendicularly to the surface of the material over a line of unit length on the same surface. It is a measure of the material’s ability to hold its molecules together in presence of a force. Given that the material of the nanobubble is known, the coefficient of surface tension for that material can be easily determined. In addition, we use an electric field threshold whose magnitude lies between the normal electric field at the neural junctions of the brain and the elevated field when epilepsy strikes. The radius of the bubble can now be calculated using the electrical permittivity of an insulating material (brain tissue), the absolute dielectric permittivity of a classical vacuum, the coefficient of surface tension of the bubble, and the electric field threshold at which the bubble should deform and rupture.

1. A nanobubble that contains epilepsy medicine which can neutralize the electric surge in the brain during the epilepsy attack.

2. A nanobubble as in claim 1 has a shell of polarizable material.

3. A nanobubble as in claim 2 with a radius and shell thickness designed for the bubble to burst above a particular threshold of electricity produced by an epileptic seizure.
